(19) United States
(12) Patent Application Publication
(10) Pub. No.: US 2012/0156794 A1
(43) Pub. Date: Jun. 21, 2012

(54) METHOD FOR THE EXTRACTION AND DETECTION OF FAT-SOLUBLE COMPONENTS FROM BIOLOGICAL MATERIALS

(76) Inventor: Florian SCHWEIGERT, Berlin (DE)

(21) Appl. No.: 13/102,653

(22) Filed: May 6, 2011

Related U.S. Application Data
(63) Continuation of application No. 12/922,973, now abandoned.

(30) Foreign Application Priority Data
Mar. 19, 2008 (EP) 08005142.8

(51) Int. Cl. G01N 21/25; C07H 3/02; C07H 1/06; C07C 45/78; C07C 7/10; C07H 3/04; G01N 21/64; C07F 9/141

(52) U.S. Cl. 436/128; 422/82.05; 436/139; 436/172; 536/18.5; 554/79; 554/83; 560/195; 568/366; 585/833

(57) ABSTRACT

The invention relates to a method for analysis of fat-soluble components, in particular fat-soluble dyes, from biological materials, in particular foods and feeds, having facilitated extraction of the fat-soluble components from the biological materials with use of suitable dilution solutions and of the extractability using pertinent organic solvents or organic solvent mixtures and also an enrichment and separation method, with subsequent digital evaluation and documentation. It is proposed to treat the biological materials first with a dilution medium which makes the fat-soluble components more readily extractable from the complex biological matrix and subsequently with at least one organic solvent which extracts the components; the substances extracted into the organic supernatant are subsequently chromatographically enriched and separated and then visually assessed and/or measured.

### FIELD

The present invention relates to a method for the analysis of fat-soluble components, in particular dyes, from biological materials, in particular foodstuffs, having an enrichment of the components and subsequent analysis. The method comprises, in particular, a combination of extraction and separation steps and a subsequent analysis step. The invention further relates to analysis kits and analytical equipment for carrying out the method.

### BACKGROUND

The method according to the invention is composed of a plurality of steps. The two steps which are essential and characterize the invention are:
Taking up a sample of the biological material into a solvent which facilitates the extractability of fat-soluble components (sample preparation step).
Extracting the fat-soluble components into an extraction mixture by means of an organic solvent or solvent mixture with simultaneous removal of the non-fat-soluble components and also optimization of the extraction relationship of naturally occurring components and the fat-soluble components to be investigation (extraction step).

Depending on the substance to be analyzed, the extracted component can be further separated before analysis.

In the foreground of the invention are the dissolution of the complex compound between the fat-soluble components and the biological matrix, provision of analytical kits and the use of the method for determining the substances which are hereinafter also called components in food and feeds.

Fat-soluble components which come into consideration for the detection method according to the invention are lipids and lipoids. This group of substances is characterized by its lack of solubility in water or aqueous solutions and its good solubility in organic solvents. This is known as lipophilicity. These substances include, in addition to the essential fats such as triglycerides, phospholipids and cholesterol, a multiplicity of substances some of which may be encountered in very small amounts and differ very greatly with respect to their chemical structure. These include fat-soluble vitamins such as retinoids, vitamin E, vitamin D and vitamin K. Other substances are steroids such as, for example, hormones, pherohormones and mycotoxins. Other substances which come into consideration for the detection method are lipophilic dyes of natural or synthetic origin. This relates in particular to the group of carotenoids (beta-carotene, alpha-carotene, canthaxanthin, astaxanthin, lutein, lycopene) as constituent of numerous plant or animal products or synthetic (artificial) dyes of the azo group (Sudan G, Sudan Brown, Sudan R, Citrus Red No. 2, Sudan Yellow GRN, Sudan II, Oil Red O, Quinoline Yellow SS, Alizarin Violet 3B, Solvent Blue 35, Quinizarine Green SS).

Natural and synthetic dyes are used for dyeing products of varying application. They serve for imparting certain optical quality features. Biological materials which can be dyed are egg yolks and egg products, spices, spice mixtures and spice preparations in solid pasty or liquid form, meat and meat products, fish and fish products, fruit and vegetable juices and/or preparations and also butter or other milk products. The dyeing of biological materials which are used for human consumption can be introduced either via the feed or in the processing.

Dyed products relate not only to foods and feeds, but also industrial products and, for example, cosmetic products.

Lipids and lipoids are associated in biological membranes or bound to specific or unspecific proteins. An example is the enrichment of lipids or lipoids such as vitamin E or phospholipids in cell membranes and is described for cells of animals and humans. This binding and the lipophilicity make particular demands of the extraction. Also, the incorporation in cell membranes or other cell structures in animal or plant biological matrices represents an extraction problem.

Modern methods for the analysis of biological materials frequently comprise steps for separation, extraction, isolation and/or enrichment of constituents and also components of the biological materials. Such method steps are indispensable both in qualitative and quantitative analysis for separating off substances which interfere or which falsify results.

The known detection methods are moreover time-consuming and costly in terms of apparatus, especially when very small amounts of components must be detected. On the other hand, substantial simplifications of the known methods generally lead to only very large concentrations being able to be detected.

Increased requirements of the safety of foods and feeds and also for the detection of toxic substances or falsifying substances, especially in international trade of goods, require rapid, sensitive and reliable analyses, preferably as close as possible to the product. Therefore, methods which consist of as few working steps as possible and/or have little technical complexity are required. In this case the use of methods which can be applied on the basis of disposable analytical kits is desirable.

In this context there is, for example, the detection of color falsification of foods and feeds with Sudan Red or the detection of likewise toxic mycotoxins.

The purpose of the invention is therefore to provide a method for the extraction of endogenous or exogenous fat-soluble substances which is selective enough to dissolve the substances with comparatively little complexity from biological material, for example a complex solid matrix, and if required can be applied simply and rapidly by means of a disposable analytical kit. At the same time, using a dilution buffer, as many differing extraction problems as possible must be able to be solved, in such a manner that the method is used, for example, for the detection of the concentration of lipids and lipophilic substances of natural or synthetic origin.

This purpose is achieved by the embodiments as described and claimed herein.

### BRIEF DESCRIPTION OF THE DRAWINGS

FIGS. 1a and 1b are graphs showing the effect of various buffers, solvents and urea on the extractability of astaxanthin from salmon muscles, with a comparison between FIG. 1a and FIG. 1b showing the effect of fat concentration of the starting sample;

FIG. 2 is a graph showing the effect of differing dilution solutions consisting of salts, buffers or urea and water on the extraction efficiency of beta-carotene in a stabilized matrix as a function of the time of incubation;

FIG. 3 is a graph showing the effect of differing preparation buffers on the extraction of Sudan Red from egg yolk into an organic supernatant of n-hexane;

FIG. 4 shows the structure of a disposal analytical kit for enrichment by extraction and subsequent enrichment and separation of fat-soluble components consisting of the extraction unit, the collecting unit and the miniaturized chromatography column; and

FIG. 5 shows the visually recognizable coloring of the stationary phase by carotenoids (arrow A) and Sudan Red (arrow B) after direct elution with the organic extraction phase from FIG. 3 (n-hexane) as mobile phase.

### DETAILED DESCRIPTION

The method as claimed in the present invention is intended for the analysis of fat-soluble components, i.e. lipids and/or lipoids, preferably natural and synthetic lipophilic components such as carotenoids, vitamins, hormones, dyes or mycotoxins from solid biological materials. In particular, the method serves for extraction of lipophilic components. The biological materials, according to the invention, are first pretreated in such a manner that the lipophilic substances are easily removed from their bound form in the aqueous environment. They can then be treated with organic solvents. In this case the components are transferred into the extraction medium and extracted.

Pretreatment, extraction and any subsequent separation are combined according to the invention in such a manner that the substances can be detected in a very small amount. Limit values which have been achieved to date only via HPLC can in this manner be achieved by the combination of very simple separation and extraction methods.

The pretreatment used is a specific dilution step (by means of a salt or buffer solution) which if appropriate, i.e. especially in the case of solid biological materials, is additionally combined with a disruption step. Disruptions which come into consideration are treatments with homogenizers. Homogenizers which may be mentioned are, for example, cutters, Ultraturrax instruments, mills etc. The biological materials are subjected hereby to a comminution in order to increase their surface area. The comminution increases the efficiency of the extraction. In addition, treatments with cold, in particular with liquid nitrogen, also come into consideration. The disruptions act to release components from the biological materials and thereby make them accessible to an extraction. The biological materials can, as pretreatment, also be subjected to a removal of impurities and/or interfering substances, in particular wash processes, for example with buffer solutions.

Surprisingly it has been found that by using various solutions such as, for example, buffer solutions or salt solutions, the extraction efficiency of lipids and lipophilic substances into an organic solvent is markedly improved compared with pure water. This is due, inter alia, to a more efficient dissolution of the complex matrix of biological materials.

The present invention therefore solves its underlying technical problem by using various buffer solutions or salt solutions for the dissolution of complex matrix structures in the context of sample preparation for a later more efficient extraction of the fat-soluble components. The method described improves the efficiency of extraction especially of fat-soluble components from various biological materials, preferably biological materials which are used in the nutrition of humans and animals.

In the context of the present invention, “biological materials” are taken to mean materials obtained from plants, animals or microorganisms, in particular endogenous materials, preferably samples of endogenous materials.

The endogenous material is preferably material which was taken from the living organism, but post-mortem removal is also possible according to the invention. In a preferred embodiment, the endogenous materials are body fluids, tissue and/or organs. These can be the body fluids blood, plasma, serum, urea, amniotic fluid, milk, uterine fluid, follicular fluid, liquor, synovial fluid, tear fluid, pancreatic secretion, gastric juice and/or saliva and also all other body fluids of physiological or pathological nature. The body fluids can be obtained by puncture and/or by means of established collection methods from the living organism or post-mortem. The blood in this case can be obtained, e.g. by puncture of blood vessels (venepuncture). Blood can be withdrawn, and plasma and serum and also other body fluids can be obtained by established methods. They comprise the puncture of vessels, separation of the blood constituents, preferably by centrifugation, and storage, preferably at −80° C. In the case of direct analysis, the sample must preferably be stored at 4° C. until analysis. Tissue and organs are taken by established biopsy methods or post-mortem and stored by corresponding established methods. Before analysis, the organs and tissues must first be homogenized, for example by established methods in the corresponding buffers. A suitable buffer is, for example, tris-HCl (pH 7.8). Methods for producing tissue homogenates are known to those skilled in the art.

“Microorganisms” in the context of the present invention, in addition to, for example, eukaryotes, such as algae, prokaryotes and/or fungi, such as yeasts, are also taken to mean viruses. In particular, “biological materials” comprise organisms obtained from culture and also culture supernatants.

According to the invention, all biological materials can be used which can be supplied to the extraction.

In a particularly preferred embodiment of the method, as biological materials, use is made of foods and feeds of animal and/or plant origin, in particular homogenates of foods.

Preferably, for the method according to the invention, foods come into consideration which comprise components which are essential for the nutrition of humans, preferably lipophilic components.

Products which originate from animals or plants, in the context of the present invention, are, for example, egg yolks and egg products, spices, spice mixtures and spice preparations in solid, pasty or liquid form, meat and meat products, fish and fish products, fruit and vegetable juices and/or preparations and also butter or other milk products.

Preferably, use is also made of those biological materials which are used in the medical sector for diagnosis and/or pursuant of a therapy.

In a further preferred embodiment of the method, the body fluids used are blood, plasma, serum, urea, amniotic fluid, uterine fluid, follicular fluid, synovial fluid, sperm, pulmonary fluid and/or secretions.

Preferably, the secretions are milk, sweat, tear fluid, saliva and/or gastrointestinal tract secretions, in particular bile fluids and/or pancreatic secretion. According to the invention, the body fluid used can be blood, preferably whole blood. Preferably, the biological material used in the method according to the invention is blood. As pretreatment, the blood is preferably treated with anticoagulants, in particular with polyanionic polysaccharides, preferably with heparin and/or heparinoids. In addition, as anticoagulants, use can be made of antithrombin III, in particular in the form of heparin-anti-thrombin complexes. In addition, anticoagulants which are foreign to the body can also be used. Anticoagulants which are foreign to the body which come into consideration are, in particular, vitamin K antagonists or calcium complexing agents. Vitamin K antagonists which may be mentioned are, in particular, cumarins, and calcium complexing agents which may be mentioned are, in particular, citrate, oxalate, preferably ethylenediamine tetraacetate (EDTA).

Advantageously, by means of the method according to the invention, a hemolysis of the blood, in particular the blood cells, is avoided. By means of the solidification according to the invention of the blood, the blood cells are embedded in a gel-like environment and thereby protected against hemolysis.

In a further embodiment, the biological materials comprise plant, animal, human and/or microbial materials which, in particular, originate from cell cultures.

In a further preferred embodiment of the method, the biological materials, before their use, are subjected to mechanical disruptions and purification processes.

For provision of the biological materials, in the method according to the invention samples of the biological materials are used, preferably samples taken from humans or animal organisms. Expediently, biological materials which are given off or secreted can also be used. In addition, it is also possible to culture the biological materials, in particular outside the human or animal body, before use of the biological materials in the method according to the invention.

It is further advantageous that the extraction is carried out manually by shaking, in particular careful shaking avoiding hemolysis. For the extraction of the blood, together with the solvents, use can also be made of aids, in particular shaking tables, pivoting rockers, overhead shakers, magnetic stirrers and other stirring techniques. Manual mixing has the advantage of being able to perform the extraction independently of a power source or of electrical instruments.

The purpose of adding a dilution solution during sample preparation is not only dilution of the sample, but especially preparation for the extraction by dissolution or solubilization of the complex matrix. Optimally, the dilution solution modifies the sample in such a manner that components can be extracted more easily, in a targeted manner and more completely. In this process, for example protein interactions with the components of the dilution solution play a role. This action is based either on a general increase of ionic concentration compared with pure water or the introduction of specific components which react with components of the matrix. This relates to the dissolving of disulfide bonds, the unfolding of proteins by strongly hygroscopic molecules or the interaction with phosphate groups. The dissolution of complex chemical structures by enzymes is also possible in the context of the inventive step.

Surprisingly, it has been found that a pretreatment with certain salt solutions or buffer solutions, in particular with differing concentrations of urea solutions, leads to a marked improvement of the complex structures of a biological matrix and thereby a more efficient extraction.

Salt solutions and buffer solutions of the solutions of organic compounds can, in the context of the present invention, either consist of only one component, or of a mixture of at least two different components.

An addition of water-soluble organic components, detergents, surfactants, in particular nonionic surfactants or emulsifiers, can also promote extraction efficiency. Additives which come into consideration are, for example, DMSO or DTT.

Also, an addition of enzymes such as, for example, proteases or lipases, is provided in order to reduce the complexity of the matrix—such as in the case of lipases, for example—or in order to remove interfering accompanying components—such as, for example, by the lipases.

When urea is used in a concentration range from 0.1 to 8 M, preferably 1 to 8 M, and especially 2 to 4 M, in the case of biological materials such as eggs, fish muscles or liver, either only a simple shaking or the use of a hand mixer of the speed of rotation and power development of a milk foamer is sufficient. As a result, complex extraction methods can be markedly improved and facilitated.

Owing to different physicochemical properties of fat-soluble components and the great differences in the matrix of the biological sample, the respective composition of a solvent for dissolution of the sample matrix is of great importance for subsequent extraction.

Further solvents for sample preparation and/or sample dilution may be described and/or defined as follows depending on the biological material used:

Buffers, in the context of the present invention, comprise a buffer solution and/or a buffer system, i.e. a mixture of substances, the pH of which (concentration of hydrogen ions), on addition of an acid or base, changes significantly less than would be the case in a unbuffered system. Such buffer solutions contain a mixture of a weak acid and its conjugate base (or of the respective salt).

Ampholytes and bifunctional molecules can also act as buffers. The factor determining the pH is the ratio or protolysis equilibrium of the buffer pair.

Examples of buffer solutions are: acetic acid/acetate buffer, phosphate buffer KH₂PO₄+Na₂HPO₄; veronal-acetate buffer of Michaelis; ammonia buffer NH₃+H₂O+NH₄Cl; HEPES (4-(2-hydroxyethyl)-1-piperazinethanesulfonic acid) PBS buffer; MES (2-(N-morpholino)ethanesulfonic acid).

Salt solutions are solutions of salts which are made up of positively charged ions, called cations, and negatively charged ions, called anions. Salts can be of organic or inorganic nature. In the narrowest sense salt is taken to mean sodium chloride (NaCl, common salt). In the broad sense, all compounds are called salts that are made up, like NaCl, of anions and cations.

Salts are termed complex salts where independent (stable) ions are present with the interaction of molecules.

In addition to salts having one type of cations, salts having two different cations are also known. These salts are termed double salts, such as alauns having the general composition M^{I}M^{III}(SO₄)₂. One example is aluminum potassium sulfate dodecahydrate (KAl(SO₄)₂.12H₂O).

In addition to the inorganic salts described, there are also salts of organic compounds. The anions of these salts originate from organic acids. Of importance here are the salts of carboxylic acids such as, for example, acetic acid, of which many salts, called acetates (CH₃COO⁻), are known. Examples are the salts sodium citrate and calcium citrate.

“Organic compounds which perform a dissolution of complex matrices” in the context of the present invention include, for example, urea. Urea originates from protein and amino acid metabolism of humans and animals. Urea, because of its high water binding capacity, is used as a keratolytic which dissolves complex matrices. It is added to foods as a stabilizer. In the EU, as a food additive with the designation E 927b, it is permitted solely for chewing gum without sugar addition.

As organic solvents for extraction and if necessary subsequent analysis, use is made of polar protic solvents, in particular alcohols.

In a further preferred embodiment of the method, use is made of the polar protic solvents selected from the group consisting of methanol, ethanol, 1-propanol, 2-propanol (isopropanol), butanol, pentanol, hexanol and also mixtures thereof. Preferably, use is made here of mixtures of ethanol and 2-propanol (isopropanol).

In a further preferred embodiment of the method, as organic solvent, use is made of at least one polar aprotic solvent, in particular esters, preferably ethyl acetate.

Advantageously, in the method according to the invention, in addition to the preferred polar protic solvents, use may also be made of polar aprotic solvents.

In a further embodiment of the invention, as polar aprotic solvents, use is made of nitriles, preferably acetonitrile.

In a further embodiment of the method, as polar aprotic solvents, use is made of ketones, preferably acetone.

In a further embodiment of the method, as polar aprotic solvents, use is made of dimethyl sulfoxide and/or N,N-dimethylformamide.

In a further embodiment of the method, as polar aprotic solvents, use is made of ethers, in particular diethyl ether.

Preferably, the polar solvents are used in the form of mixtures depending on the biological materials.

In a further preferred embodiment of the extraction step, as solvents, use is made of at least one nonpolar solvent, in particular alkanes, preferably C5 to C12 alkanes.

In a further preferred embodiment of the extraction step, as nonpolar solvent, use is made of hexane, heptane and/or octane, in particular isooctane

In a further embodiment of the extraction step, as nonpolar solvents, use is made of aromatics, in particular toluene and/or benzene.

Said nonpolar solvents act in the method according to the invention as extraction media for the components, in particular for the lipophilic components.

According to the invention it can be expedient to use derivatives of organic solvent molecules. The solvents can be anhydrous, water-containing, branched, unbranched, cyclic, acyclic, halogenated or nonhalogenated.

Division into polar or nonpolar solvents can be performed in industry from various aspects. For example, definitions of polarity or solvent behavior known from chemistry can be used.

In addition, a polarity index according to Snyder or Keller is used in practice (Synder, Principles of absorption chromatography, Decker, New York, 1968; Keller, Analytical chemistry, Weinheim, 1998, page 195), for classifying solvents or solvent mixtures. According to this, polar solvents or solvent mixtures are taken to mean a solvent or solvent mixture having a polarity index of 4 to 8, in particular 5 to 7, preferably 5.5 to 6.5, according to Snyder. Polar solvents are, for example, water, in particular aqueous solutions. Polar aprotic solvents are, for example, acetone, acetonitrile, ethyl acetate, dimethyl sulfoxide or N,N-dimethylformamide. Polar protic solvents are, for example, alcohols which comprise an alkyl moiety having 1 to 6 carbon atoms, for example methanol, ethanol, 1-propanol, 2-propanol (isopropanol), butanol, pentanol or hexanol.

A nonpolar solvent or nonpolar solvent mixture is taken to mean a solvent or solvent mixture which, in comparison with a reference solvent or reference solvent mixture, has a polarity index which is 0.3 or more lower. Preference is given to a polarity index which is 0.5 lower, in particular a polarity index 1 lower, preferably a polarity index lower by more than 2. Consequently, the polarity index of the nonpolar solvent or solvent mixture has a value of 5 to 1, in particular 4 to 2, preferably 3.5 to 2.5, according to Snyder. A solvent mixture of 60% methanol/40% dichloromethane has, for example, a polarity index of 3.1 according to Snyder. Nonpolar solvents which consequently come into consideration are, for example, halogenated solvents such as chloroform, dichloromethane or carbon tetrachloride. In addition, aliphatic solvents such as pentane, hexane, heptane or cyclohexane may be mentioned. In addition, nonpolar solvents which may be mentioned are aromatic solvents such as toluene or benzene.

Furthermore, ethers such as diethyl ether, tert-butyl methyl ether or tetrahydrofuran come into consideration.

In a further preferred embodiment of the method according to the invention, the solvents of the extraction step are used in the form of solvent mixtures, in particular in the form of solvent mixtures which comprise polar and nonpolar solvents.

Preferably, the polar and nonpolar solvents are used in a ratio of 1:1, in particular in a ratio of 1:2, preferably in a ratio of 1:10 (polar:nonpolar).

This measure has the advantage that solvent mixtures can be produced which are matched according to the properties of the biological materials. In this manner, a multiplicity of separation problems can be handled.

In the necessary enrichment and separation steps, for example by miniaturized chromatographic methods, which are subsequent to the extraction, it can be expedient to select the composition of the extraction solvents in such a manner that no mixture and/or complete separation of, for example, alcohols and organic solvents, occurs. A suitable solvent here is preferably DMSO (dimethyl sulfoxide) as aprotic dipolar solvent and n-hexane or isooctane. In this case the nonpolar solvent can also be added to the processing buffer. As a result the solvent which is extracting can equally be used as mobile solvent for the subsequent chromatographic enrichment and separation.

In a further preferred embodiment of the extraction step, in addition use is made of surfactants, in particular nonionic surfactants.

In a further preferred embodiment of the extraction step, as surfactants, use is made of copolymers, in particular copolymers of poly(ethylene oxide)s and poly(propylene oxide)s.

Preferably, use is made of those surfactants which, in the solvent mixture, are soluble, free of toxic properties and/or leave unaffected the analysis of components situated in the supernatant. Preferably, absorption and/or fluorescence of the components remain measurable in an unimpaired manner.

Preferably, the surfactants are used in concentrations which exclude the hemolysis of blood. Advantageously, as surfactants, use is made of copolymers, in particular copolymers of poly(ethylene oxide)s and poly(propylene oxide)s. As an example of copolymer surfactants, commercially available Pluronic surfactants come into consideration, preferably Pluronic 101.

In a further preferred embodiment, the biological materials are treated with the organic solvents in a ratio of 1:50, in particular in a ratio of 1:10, preferably in a ratio of 1:3.

Depending on the properties of the biological materials and extraction capacity of the organic solvents, it can be expedient to select a greater or lesser ratio of biological materials to organic solvents, and in particular this depends on the subsequent analysis. Preferably, the ratio is selected in such a manner that the detection limit or limit of determination in the analysis of the components is taken into account.

In a further embodiment, the method is carried out at a temperature in the range from 5° C. to 60° C., in particular in the range from 10° C. to 40° C.

In a further embodiment, the method is carried out at a pressure between 0.5 bar and 5 bar, in particular between 0.8 bar and 2 bar.

In theory the method may be carried out in temperature and/or pressure ranges at which gel formation may be expected. In addition, solvent properties, in particular melting point, boiling point, flash point must be taken into account. According to the invention the method is carried out at room temperature and atmospheric pressure.

In a further preferred embodiment of the method, the biological materials are treated for extraction of the components for a time period of from 10 seconds to 10 minutes, in particular from 10 seconds to 5 minutes, preferably from 10 seconds to 3 minutes.

Preferably, the samples are pretreated with a dilution buffer before the extraction. The dilution ratio is between 1:9 (buffer:sample) and 100:1, in particular 1:1 to 50:1, preferably 10:1.

Preferably, the biological materials are transferred into a solvent-resistant environment before the treatment with the organic solvents.

In a further preferred embodiment, as solvent-resistant surrounds, use is made of a vessel having a hydrophobic surface, in particular a vessel having a surface which is hydrophobized by silanization.

In a further embodiment, use is made of a vessel having a hydrophobic surface, in particular a plastic vessel, preferably made of polypropylene.

Advantageously, in the method according to the invention, vessels having hydrophobic surfaces can be used. The hydrophobic surfaces, in the case of glass vessels, may be produced by silanizing the glass surface or by etching it with hydrogen fluoride. In addition, plastic vessels, preferably made of polypropylene, can also be used in the method according to the invention. A use of composite materials for the vessels, in particular plastic-coated vessels, is also possible. Preferably, use is made of vessels which are of a nature such that they are suitable for spectroscopic examinations.

In a further preferred embodiment of the method, the components, before the extraction, are transferred to a lipophilic form and/or modified so as to be lipophilic.

The invention further relates to methods of analyzing the extracted components. For analysis of the components, all known analytical techniques or else analytical techniques which are unknown to date come into consideration. Separation of the components, for example by chromatographic methods, in particular by high performance liquid chromatography (HPLC) can prove to be required for further analysis. Expediently, the supernatant is supplied to analytical methods which examine the components spectrometrically. Spectrometric methods which come into consideration are those which examine the components by an interaction with electromagnetic radiation, in particular NMR, IR, UV-VIS, laser-Raman spectroscopy. In addition, all known mass spectrometric methods can be used.

A preferred embodiment of the analysis proceeds via a spectrophotometer. Particular preference is given to a handleable transportable instrument with which the photometric measurement can be carried out rapidly and reliably.

The invention further relates to analytical units containing organic solvents or solvent mixtures and the use thereof for the direct analysis of extracted substances.

Particularly preferred analytical units consist of two separate analytical kits, namely a pretreatment kit and an extraction kit, with which the method according to the invention can be carried out in two steps. For instance the biological material is disrupted and diluted in a pretreatment kit. Subsequently a liquid sample is transferred from the pretreatment kit into an extraction kit in which the substance is then extracted, transferred to the organic phase and measured directly with the spectrophotometer.

Both kits are formed in this case, for example, by one vessel each which is formed of plastic or glass and which contains the buffer medium and/or solvent dependent on the biological material and the substance to be analyzed.

Depending on the properties of the components, the latter can be further worked up before analysis, for example by enrichment and/or separation on miniaturized capillaries which are packed with separation materials.

In a further preferred embodiment of the analytical method, the components dissolved in the organic solvent are directly enriched and simultaneously separated. The separation can be performed using complex chromatographic methods such as HPLC or gas chromatography, or via standard or miniaturized column chromatography or thin-layer chromatography. In the preferred embodiment, the components are enriched directly from the extraction unit under pressure on a miniaturized chromatography column and separated from interfering substances. The enrichment and separation is an absorption method. In this case the substances are retained on the stationary phase by Van der Waals' forces, dipole-dipole interactions or hydrogen bonds.

In the preferred embodiment, enrichment and separation proceed on the stationary phase via the same solvent or solvent mixture which was used for the extraction.

However, a stepwise enrichment and separation is also possible using different solvents or solvent mixtures.

In the preferred embodiment, the lipophilic components which are to be separated and examined, are, after the extraction step, already in the mobile phase.

The enrichment and/or separation proceeds by means of a stationary phase. Stationary phases which come into consideration are silica gel, cellulose, cyclodextrin, aluminum oxide, florisil and other substances which, owing to their physicochemical properties, are suitable for the respective component. The selection of mobile and stationary phases depends on the separation problem. The materials can, in addition, be modified in their surface properties by targeted chemical modifications. As an example of the chromatography of lipids, the surface treatment of silica gels with silver ions may be mentioned. However other methods can also be suitable.

In addition to the use of uniform stationary phases, stationary phases can also be combined. This combination can proceed either by mixing a plurality of different phases or by a layerwise structure of the column packing. By means of the layerwise structure, various separation and enrichment effects can be achieved.

The pressure buildup for the flow of the mobile phase can be generated either via gravity or via other methods by which a low pressure causes the mobile phase to run.

In the preferred embodiment, the flow of the mobile phase is generated by the one opening of a miniaturized column packed with the stationary phase being brought into the closed extraction unit. This proceeds by penetration of a rubber septum or a septum of another kind. A spacer on the column defines the depth of penetration of the column into the extraction tube. As a result, at constant sample volume and constant solvent volume it can be ensured that the opening of the column is in the organic extraction medium and that only a defined amount of solvent is used.

The pressure for the flow of the solvent is built up by piercing using a syringe via a needle next to the chromatography tube and pumping in about 10 ml of air via the syringe. This volume is dependent on the size of the extraction tube and the volume of the solvent. By means of the overpressure which is created, flow of the mobile phase occurs.

In the preferred embodiment, a further empty extraction tube is stationed on the opposite side, in which extraction tube the mobile phase is collected. As a result no fouling of the working place occurs. The rubber septum which is likewise present closes after removal of the chromatographic column and both units can be disposed of without the examiner coming into contact with the chemicals.

Identification of the enriched and/or separated fat-soluble components proceeds either directly by eye or by spectroscopic methods if substances having a characteristic inherent color are concerned; or by means of fluorescence if the substances have characteristic excitation and emission spectra.

Examples of fat-soluble components having characteristic inherent colors may be found, for example, in the group of natural and synthetic (artificial) dyes. These include carotenoids and azo dyes. Fat-soluble substances having a characteristic inherent fluorescence are, for example, vitamin A compounds, vitamin E compounds and mycotoxins.

In a further step, the substances can also be modified in order to be able to be detected subsequently by substance-specific reactions. These modifications can proceed at various positions of the described detection method: before the organic extraction in the biological material directly, or in the material after or on uptake into the dilution buffer, in the organic extract, or during or after separation on the chromatography column.

Further details and features of the invention result from the description hereinafter of the performance of the method according to the invention and from preferred embodiments in combination with the subclaims. In this case the respective features can each be implemented alone or a plurality can be implemented in combination with one another.

### Example 1

### Determination of Carotenoids and Vitamins in Egg Yolk

Egg yolk consists to a large fraction of lipids. Coloring components are carotenoids. In eggs, there may be found, in differing relations to one another, the carotenoids lutein, zeaxanthin β-carotene and canthaxanthin. They arrive in the egg yolk via the feed. The following example indicates the effect of composition of the dilution solution on the extractability of carotenoids from eggs.

In the context of the pretreatment step, in each case 200 mg of egg yolks are mixed to make up 10 times the volume with a buffer or salt solution (NaCl solution, urea solution, phosphate buffer solution) or distilled water and subsequently isolated in a single step in a single-phase solvent mixture consisting of two different solvents. The carotenoids were determined in the organic extract either by means of HPLC or spectroscopy. Vitamins A and E were determined by means of HPLC.
a) Mixing 200 mg of egg yolk with respectively 1.8 g of dilution solution either distilled water or buffer solution or salt solution (NaCl, PBS or urea). Intense manual or mechanical mixing (pretreatment step).
b) Taking up the mixture (generally 400 μl) in a syringe and injecting it into a special cuvette via a rubber septum. Special cuvette contains a single-phase solvent mixture.
c) Extracting the fat-soluble components into the solvent mixture by intense shaking.
d) Separating the phases by sedimentation for 3 minutes.
e) Measuring the supernatant directly in the spectrophotometer (for example the portable iCheck photometer)

Alternatively taking off the solvent supernatant and measuring the components by means of HPLC or spectrophotometry.

### Example 2

### Determination of Carotenoids (Astaxanthin) in Fish Flesh

Fish muscle, depending on species, consist to a large fraction of fats of varying chemical structure. Coloring components in the fish muscle in the case of salmon and salmon trout are the carotenoids. The most important carotenoid is astaxanthin. They pass into the muscle flesh via the feed. The red coloring is an essential quality feature for consumers. The following example shows the effect of composition of the dilution solution (buffer, salt solutions, urea solution) on the extractability of carotenoids from salmon muscles.

### Steps

a) In the context of the pretreatment step, in a first step first in each case about 3 g of fish muscle tissue is comminuted by means of a press (modified garlic press). Other possible methods of comminution are also conceivable.
b) Subsequently, in each case 200 mg of comminuted tissue are taken up into 2 ml of distilled water or buffer solution consisting of, for example, an NaCl solution, urea solution or phosphate buffer solution (dilution solution) and further communited, disrupted and prepared for the subsequent extraction (pretreatment step) by intense mixing, for example using a modified milk foamer.
c) Taking up the substantially homogeneous mixture (800 μl) in a syringe having a volume of 1 ml and injecting it into a special cuvette via a rubber septum. The special cuvette contains a single-phase solvent mixture consisting generally of two different organic solvents.
d) Extracting the fat-soluble components into the solvent mixture by intense shaking.
e) Separating the phases by sedimentation over 3 minutes.
f) Measuring the supernatant directly in the spectrophotometer (for example the portable iCheck photometer)
g) Alternatively taking off the solvent supernatant and measuring the components by means of HPLC or spectrophotometry. Vitamins A and E were determined by means of HPLC.

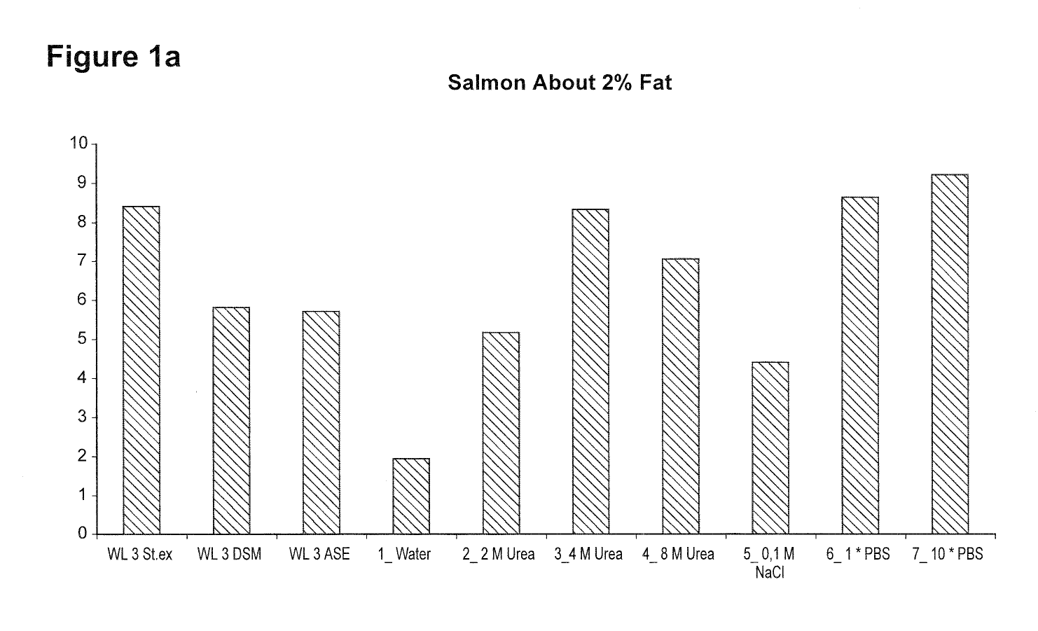

FIGS. 1a and 1b show the effect of various buffers, solvents and urea on the extractability of astaxanthin from salmon muscles. The comparison between FIGS. 1a and 1b shows the effect of fat concentration of the starting sample. The results are reported as mg/g of fish muscle. Differing results are achieved as a function of the fat concentration for comparable dilution solutions. The most uniform for both matrices was 2 to 4 M urea solution.

Abbreviations used: urea=urea solution; NaCl=sodium chloride solution;

PBS=phosphate buffer in 1 to 10 fold concentration.

### Example 3

### Stabilized Carotenoid Preparations

For improvement of the storage and processing ability of the sensitive carotenoids, these are packed in a stabilizing matrix.

The following example shows the effect of distilled water and differing buffers on the extractability of the carotenoids as a function of the incubation time.

For preparation for the extraction, a β-carotene preparation (in each case 100 mg) was incubated with various buffers, salt and urea solutions of differing concentration and distilled water (in each case 10 ml) over a time period of in total 5 hours (pretreatment step).
a) Incubation at room temperature for 30 min, 1, 2, 3, 4 and 5 hours.
b) Centrifugation of the sample for separating off the undissolved components.
c) Takeoff of the supernatant.
d) Dilution 1:100 in the starting solvent.
f) Taking up the mixture (400 μl) in a syringe (1 ml volume and injection into a special cuvette via a rubber septum. Special cuvette contains a single-phase solvent mixture.
g) Extraction of the fat-soluble components into the solvent mixture by intense shaking
h) Separation of the phases by sedimentation for 3 minutes.
i) Measuring the supernatant directly in the spectrophotometer (for example in a portable photometer)
j) Alternatively takeoff of the solvent supernatant and measuring the components by means of HPLC or spectrophotometry.

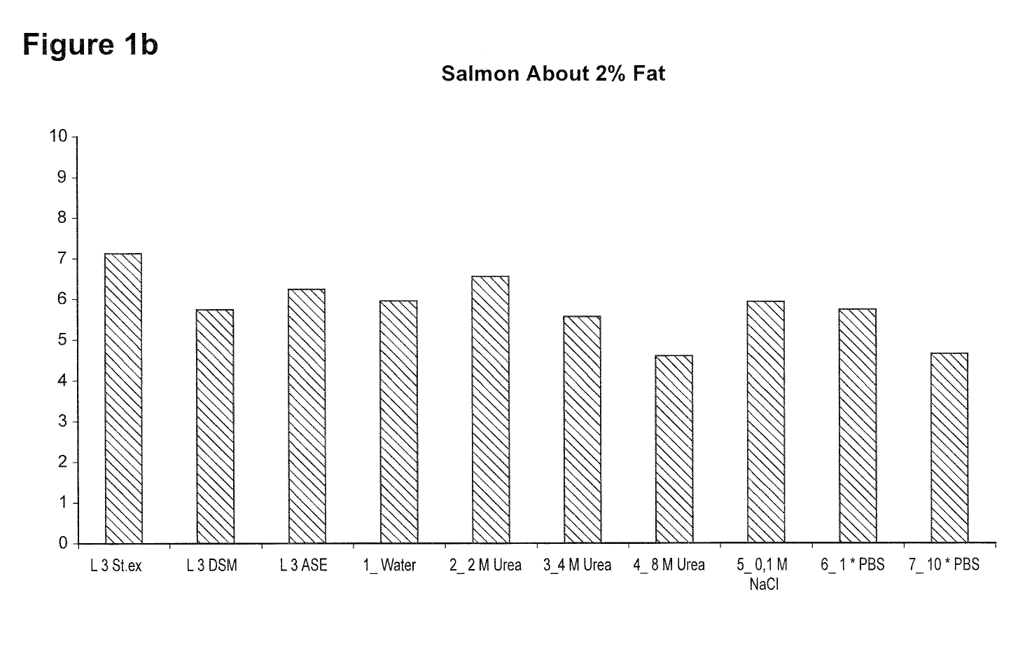

FIG. 2 shows the effect of differing dilution solutions consisting of salts, buffers or urea and water on the extraction efficiency of beta-carotene in a stabilized matrix as a function of the time of incubation. The marked efficiency of urea in differing concentrations (for example 2 or 4 M) may be seen. The results are shown as percent of the maximum achieved concentration and actual concentration.

Abbreviations used: urea=urea solution; NaCl=sodium chloride solution;

PBS=phosphate buffer in 1 to 10 fold concentration.

### Example 4

### Extraction and Determination of Sudan Red and Carotenoids from Egg Yolk

For improving the yellow coloring and for increasing the color stability, dyes are added to various naturally yellow or red colored foods. Whereas the natural or nature-identical carotenoids are safe, when azo compounds are added there is a high level of health risks. For this reason their addition is forbidden and products which contain these artificial colors must be removed from the market. Rapid reliable and sensitive detection is necessary for this purpose.

Examples of such a method are described hereinafter for egg yolk.

A large fraction of egg yolk consists of lipids. Coloring components are the carotenoids. In eggs the carotenoids lutein, zeaxanthin β-carotene and canthaxanthin are found in differing relationships to one another. They pass into the egg yolk via the feed. To intensify the coloring, the feeding of synthetic dyes of the group of azo compounds is performed. An important representative is Sudan III (red).

The following example demonstrates the steps for extraction and detection of Sudan Red from egg yolks.

In each case 200 mg of egg yolks were mixed with 10 times the volume of a buffer solution or distilled water and subsequently isolated in a single step in a single-phase solvent mixture. The carotenoids were determined in the organic extract either by means of HPLC or spectroscopy. Vitamins A and E were determined by means of HPLC.
a) Mixing of 200 mg of egg yolk with in each case 1.8 g of diluent, either distilled water or buffer solution. Intense manual or mechanical mixing.
b) Takeup of the mixture (generally 400 μl) in a syringe and injection into a special extraction unit via a rubber septum. The extraction unit contains a solvent or a single-phase solvent mixture.
c) Extraction of the fat-soluble components into the organic extraction medium by intense shaking.
d) Separation of the phases by gravity for 3 minutes.
e) Optimization of the phase separation by addition of a highly concentrated salt or buffer solution in a volume of preferably 500 μl.
f) Measurement of the supernatant directly in the spectrophotometer.
g) Alternatively takeoff of the solvent supernatant and measuring the components by means of HPLC or spectrophotometry. Vitamins A and E were determined by means of HPLC.
h) For enrichment and separation, the extraction is followed by column-chromatographic separation. For this purpose a miniaturized chromatography column packed with silica gel (stationary phase) is brought into the extraction tube. For this the rubber septum of the extraction unit is penetrated and the lower introduced end of the column positioned in such a manner that it is just above the aqueous phase. On the opposite side of the column the collecting unit is mounted.
i) By means of a syringe of 5-10 ml volume, via a needle, air is pumped via the septum into the extraction unit for approximately 10 min. An overpressure is formed which leads to the organic extraction medium flowing from the extraction unit via the chromatography column (stationary phase silica gel) into the collecting unit. In this process enrichment and separation of the carotenoids and of Sudan Red occurs.
j) Semiquantitative estimation of the concentration proceeds via comparison of the column containing the sample with columns which have a known concentration of Sudan Red.
k) Detection is performed by eye or by digital amplification of the optical signals. Carotenoids and Sudan Red are situated in separate bands.

### Example 5

### Detection of Mycotoxins

Mycotoxins are contaminants of cereals, cereal products or, for example, nuts and coffee which are infected by various fungi. Since they represent a large health risk for humans and animals
a) contaminated cereals (1 g) were admixed with 1 ml buffer solution (1 molar urea) and incubated for 10 minutes and shaken intensely manually for 10 seconds 5 times at regular intervals.
b) Takeoff of the aqueous supernatant by means of a syringe (1 ml) and injection of 800 ml into the extraction unit. The extraction unit contained a single-phase mixture of organic solvents. A mixture of polar and nonpolar solvents (ethanol/isopropanol/isooctane; 1:4:10) was used. Immediate phase separation occurs. The mixture is shaken manually carefully for 10 seconds. Subsequently phase separation occurs by sedimentation for 5 minutes. After 2 to 3 minutes, the mixture is again shaken and allowed to stand.
k) Measurement of the supernatant directly in the fluorescence photometer.
l) Alternatively takeoff of the solvent supernatant and measuring of the components by means of HPLC or fluorescence photometry.
m) For enrichment and separation, the extraction is followed by column-chromatographic separation. For this a miniaturized chromatography column packed with florisil (stationary phase) is introduced into the extraction tube. For this the rubber septum of the extraction unit is penetrated and the lower introduced end of the column positioned in such a way that it is just above the aqueous phase. On the opposite side of the column the collecting unit is mounted.
n) By means of a syringe of 5-10 ml volume, approximately 10 ml of air is pumped via a needle into the extraction unit via the septum. An overpressure is formed which leads to the organic extraction medium flowing from the extraction unit via the chromatography column (stationary phase florisil) into the collecting unit. In this process enrichment and separation of the mycotoxins occur.
o) Semiquantitative estimation of the concentration proceeds via comparison of the column containing the sample with columns which have a known concentration of mycotoxins.
p) Detection of a blue fluorescence of the mycotoxins proceeds with UV excitation by eye or by digital amplification of the optical signals.

### Example 6

### Determination of Astaxanthin-Content in Rainbow Trout Flesh by iCheck Method in Comparison with HPLC

### Material and Methods

The control treatment of the pigmentation trial CP078 in which rainbow trout were fed in triplicate tanks (A1, A2 and A3) a diet supplemented pre-extrusion with 50 ppm astaxanthin as CAROPHYLL Pink 10%-CWS. The final sampling was performed after 12 weeks of experimental feeding. Following the normal procedure for sampling in pigmentation trial, 8 fish were sampled per tank, killed, bled and filleted. The fillet was skinned and a 15 g flesh sample was taken at the level of the NQC, frozen and given to NRD/CM for HPLC astaxanthin determination according to the standard method. Another smaller flesh sample of ca 5 g was taken at the same NQC level and frozen for further iCheck determination of astaxanthin. iCheck fish prototypes for astaxanthin determination were provided by BioAnalyt for these tests. The iCheck analyses were performed at CRNA according to the protocol established by BioAnalyt which defines a sample size of 0.5 g flesh homogenate mixed into 1.5 g of buffer in the first phase (Detailed description in Annex 1). The samples were analyzed in duplicates in order to evaluate the reproducibility of the technique.

The technique was then slightly modified in order to increase the sample size and see whether this could improve the accuracy of astaxanthin determination. In this case, the sample size was increased to 0.87 g flesh homogenate which were mixed with 2.61 g buffer in order to keep the same dilution rate of the sample and to get to the maximum limit of data input of the spectrophotometer.

### Results

Tables 1 and 2 present the results of measurements of astaxanthin in trout flesh samples of 0.5 g by iCheck and HPLC, respectively.

Table 3 presents the results of astaxanthin measurements obtained when the size of the sample was increased from 0.5 g to 0.87 g for samples from fish 6 to 8 in each replicate group.

Table 4 presents the individual and average differences between HPLC and iCheck measurements expressed as %, for the two sample sizes. The variation between duplicate measurements is relatively low in most cases.

Results of astaxanthin determination are generally higher by HPLC (table 2) than by iCheck method (table 1) with samples of 0.5 g. Observation was made that in the conditions described in the iCheck procedure, the extraction of carotenoids was not complete as some flesh homogenate particles were still pigmented in the iCheck vials after three repeated shakings at 5 min time intervals. Astaxanthin recovery from iCheck extraction procedure was not complete in most cases.

Table 3 shows the results of iCheck measurements with flesh samples of 0.87 g. This was only done for three fish (fish 6 to 8) per replicate groups. Despite some variations, there is a clear trend for an improved carotenoid recovery with increased sample size. The % differences between iCheck and HPLC measurements are in the range of 20 to 30% when the size of the samples was 0.5 g (table 4) and they tend to decrease reaching 7% to 18% as average between replicate groups with samples of 0.87 g. The variations are bigger with the flesh samples of 0.87 g due to the lower number of samples analyzed.

Although the method developed for quick analysis of astaxanthin works well, improvement in terms of recovery of carotenoid through iCheck procedure are required in order to lower the % difference with HPLC to below 10% on a constant basis.

TABLE 1
Astaxanthin contents in mg/kg of trout flesh measured with iCheck using 500 mg
sample. Values represent individual measurements of 8 fish per replicate tanks in
duplicates.

| | | A1 | | A2 | | A3 | |
|---|---|---|---|---|---|---|---|
| | | Asta | | Asta | | Asta | |
| Fish | Rep. | mg/kg | Means ± SD | mg/kg | Means ± SD | mg/kg | Means ± SD |
| 1 | 1a | 6.40 | 7.65 ± 1.76 | 6.35 | 7.59 ± 1.75 | 7.70 | 8.18 ± 0.67 |
| | 1b | 8.89 | | 8.83 | | 8.65 | |
| 2 | 2a | 4.66 | 5.99 ± 1.87 | 7.15 | 7.32 ± 0.23 | 6.61 | 7.07 ± 0.65 |
| | 2b | 7.31 | | 7.48 | | 7.53 | |
| 3 | 3a | 7.37 | 7.77 ± 0.57 | 9.34 | 9.34 ± 0.00 | 8.05 | 8.32 ± 0.38 |
| | 3b | 8.17 | | 9.34 | | 8.59 | |
| 4 | 4a | 7.09 | 7.57 ± 0.68 | 7.65 | 8.56 ± 1.29 | 7.59 | 8.31 ± 1.01 |
| | 4b | 8.05 | | 9.47 | | 9.02 | |
| 5 | 5a | 9.93 | 9.70 ± 0.33 | 7.99 | 8.67 ± 0.95 | 8.83 | 9.02 ± 0.27 |
| | 5b | 9.47 | | 9.34 | | 9.21 | |
| 6 | 6a | 8.23 | 8.32 ± 0.13 | 6.82 | 7.53 ± 1.00 | 8.47 | 9.44 ± 1.37 |
| | 6b | 8.41 | | 8.23 | | 10.41 | |
| 7 | 7a | 5.80 | 6.42 ± 0.88 | 8.29 | 9.08 ± 1.11 | 9.60 | 10.01 ± 0.57 |
| | 7b | 7.04 | | 9.86 | | 10.41 | |
| 8 | 8a | 8.89 | 9.41 ± 0.74 | 10.20 | 10.41 ± 0.30 | 7.26 | 7.12 ± 0.20 |
| | 8b | 9.93 | | 10.62 | | 6.98 | |

TABLE 2
Astaxanthin content in mg/kg of trout flesh measured with HPLC.
HPLC (Asta mg/kg)

| Fish | A1 | A2 | A3 |
|---|---|---|---|
| 1 | 10.69 | 10.12 | 11.21 |
| 2 | 10.44 | 9.92 | 9.08 |
| 3 | 10.78 | 12.10 | 11.00 |
| 4 | 13.38 | 10.85 | 10.10 |
| 5 | 12.18 | 12.09 | 9.65 |
| 6 | 11.60 | 9.08 | 11.20 |
| 7 | 8.60 | 11.57 | 12.46 |
| 8 | 11.45 | 11.74 | 9.27 |
| Mean | 11.14 | 10.93 | 10.50 |
| SD | 1.40 | 1.13 | 1.16 |

Values represent individual measurements of 8 fish per replicate tanks.

TABLE 3
Astaxanthin contents of trout flesh measured with iCheck with
flesh samples of 0.87 g

| | | A1 | | A2 | | A3 | |
|---|---|---|---|---|---|---|---|
| | | Asta | | Asta | | Asta | |
| Fish | Reps | mg/kg | Means ± SD | mg/kg | Means ± SD | mg/kg | Means ± SD |
| 6 | 6a | 11.29 | 10.62 ± 0.95 | 8.42 | 8.21 ± 0.30 | 10.15 | 9.82 ± 0.47 |
| | 6b | 9.94 | | 8.00 | | 9.48 | |
| 7 | 7a | 8.66 | 8.11 ± 0.78 | 6.89 | 8.91 ± 2.85 | 8.24 | 8.18 ± 0.08 |
| | 7b | 7.55 | | 10.92 | | 8.12 | |
| 8 | 8a | 11.82 | 10.72 ± 1.56 | 10.49 | 10.53 ± 0.05 | 8.30 | 9.91 ± 2.27 |
| | 8b | 9.61 | | 10.56 | | 11.51 | |

TABLE 4
Percentages of differences between the results of HPLC and
iCheck methods for the two flesh sample sizes (HPLC = 100%)

| | A1 | | A2 | | A3 | |
|---|---|---|---|---|---|---|
| | 0.5 g | 0.87 g | 0.5 g | 0.87 g | 0.5 g | 0.87 g |
| Fish | (%) | (%) | (%) | (%) | (%) | (%) |
| 1 | 28.44 | — | 25.00 | — | 27.03 | — |
| 2 | 42.62 | — | 26.21 | — | 22.14 | — |
| 3 | 27.92 | — | 22.81 | — | 24.36 | — |
| 4 | 43.42 | — | 21.11 | — | 17.72 | — |
| 5 | 20.36 | — | 28.29 | — | 6.53 | — |
| 6 | 28.28 | 8.45 | 17.07 | 9.58 | 15.71 | 12.32 |
| 7 | 25.35 | 5.70 | 21.52 | 22.99 | 19.66 | 34.35 |
| 8 | 17.82 | 6.38 | 11.33 | 10.31 | 23.19 | −6.90 |
| Mean | 29.28 | 6.84 | 21.67 | 14.29 | 19.54 | 17.86 |
| SD | 9.32 | 1.43 | 5.41 | 7.54 | 6.40 | 14.54 |

### Further Figures, Descriptions

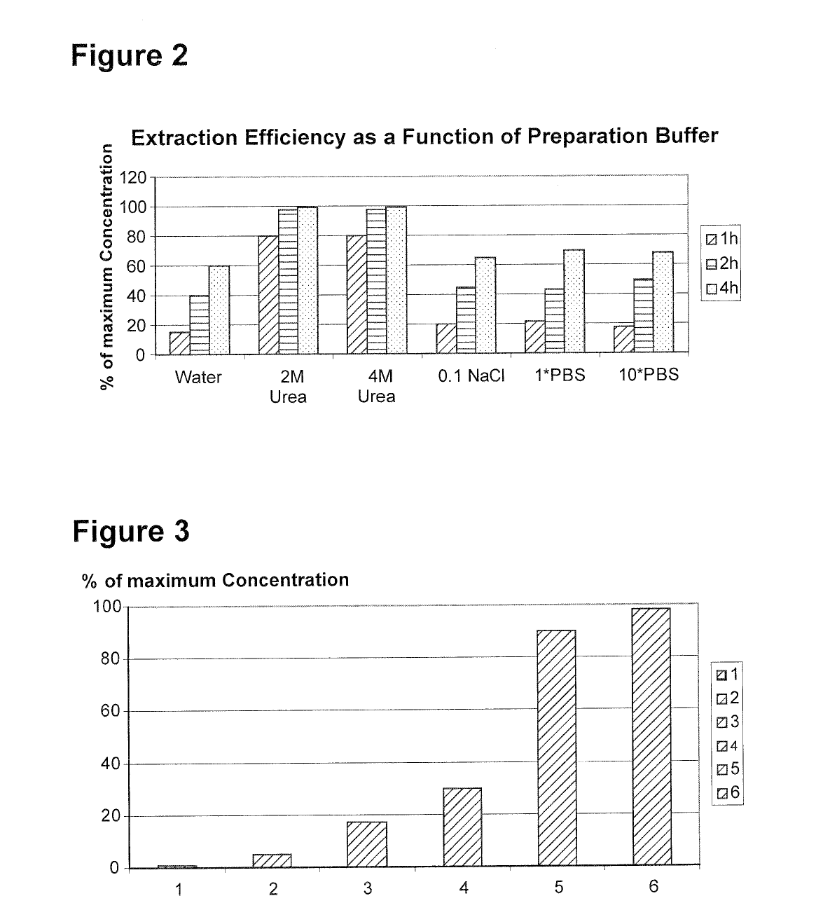

FIG. 3 shows the effect of differing preparation buffers on the extraction of Sudan Red from egg yolk into an organic supernatant of n-hexane. The following apply:

1=water (dilution solution); n-hexane (extraction solvent)

2=10% DMSO+1 M urea (dilution solution); n-hexane (extraction solvent)

3=25% DMSO+1 M urea (dilution solution); n-hexane (extraction solvent)

4=50% DMSO+1 M urea (dilution solution); n-hexane (extraction solvent)

5=75% DMSO+1 M urea (dilution solution); n-hexane (extraction solvent)

6=1 M urea (dilution solution); ethanol:isopropanol:n-hexane (volume fractions 1:2:6) (extraction solvent)

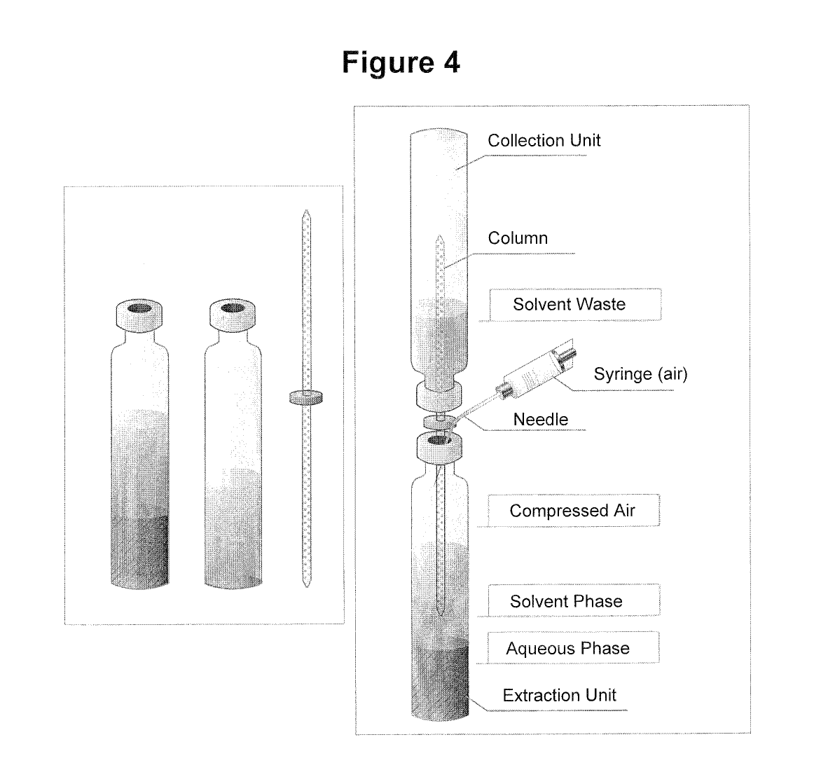

FIG. 4 shows the structure of the disposal analytical kits for enrichment by extraction and subsequent enrichment and separation of fat-soluble components consisting of the extraction unit, the collecting unit and the miniaturized chromatography column.

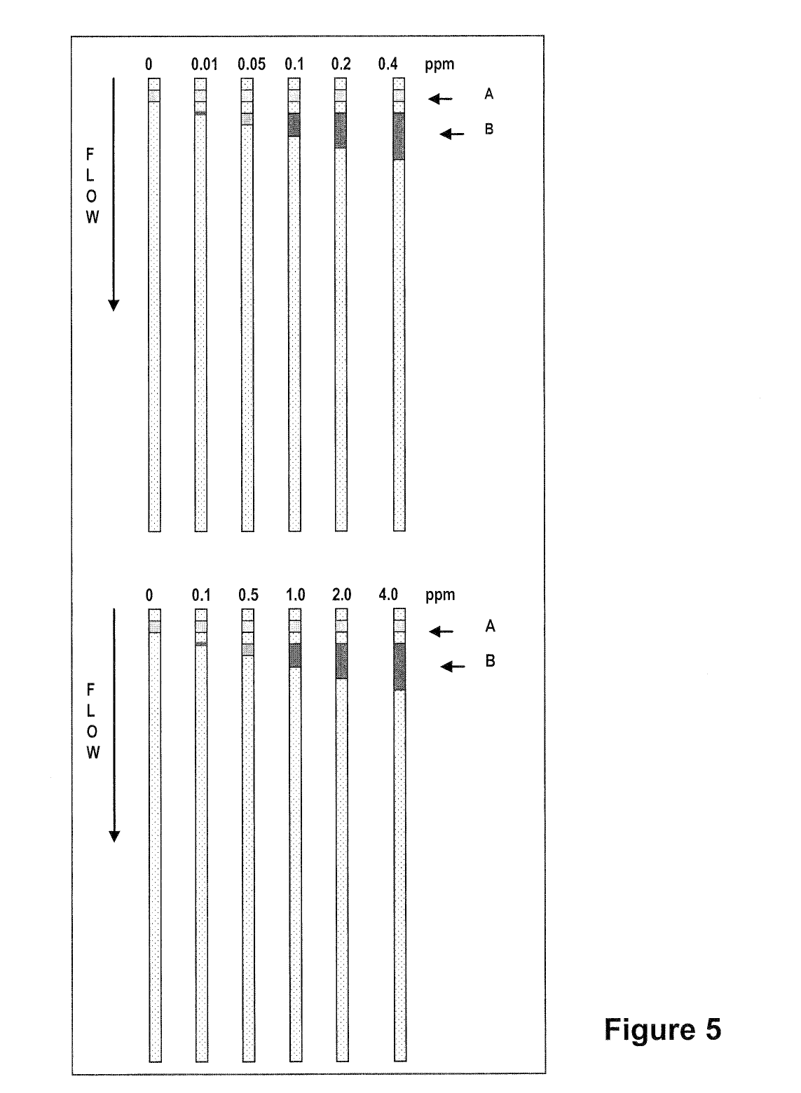

FIG. 5 shows the visually recognizable coloring of the stationary phase by carotenoids (arrow A) and Sudan Red (arrow B) after direct elution with the organic extraction phase from FIG. 3 (n-hexane) as mobile phase. Comparison of FIGS. 4a and 4b shows the possibility of signal amplification by means of digitalization. In FIG. 4b, a digital amplification of the optical signals was carried out.

1. A method for the extraction of components, in particular lipids and lipoid substances, from biological material, which comprises a pretreatment step which acts to make the component more readily available to the extraction and a subsequent extraction into an organic solvent consisting of a single-phase solvent mixture which, as a result of the addition of the aqueous sample, divides into two phases and the components can be detected in the organic solvent phase.

2. The method as claimed in claim 1, wherein the lipids, lipoids or fat-soluble substance classes are fat-soluble vitamins, fat-soluble hormones, pherohormones, or mycotoxins.

3. The method as claimed in claim 1, wherein the lipophilic substances are monogalactosyl diacylglyceride, cardiolipin, digalactosyl diacylglyceride, phosphatic acid, phosphatidylcholine, phosphatidylethanolamine, phosphatidyl-D-L-glycerol, phosphatidylinositol or phosphatidyl-L-serine.

4. The method as claimed in claim 1, wherein the lipophilic substances are natural or synthetic (artificial) fat-soluble dyes.

5. The method as claimed in claim 4, wherein the dyes are carotenoids such as, for instance, astaxanthin, canthaxanthin, beta-carotenes or apo-esters.

6. The method as claimed in claim 4, wherein the synthetic dyes are azo compounds.

7. The method as claimed in one of the preceding claims, wherein the biological materials used are foods or feeds of animal and/or plant origin, in particular homogenates of foods or feeds.

8. The method as claimed in one of the preceding claims, wherein the endogenous materials are body fluids, tissue and organs.

9. The method as claimed in one of the preceding claims, wherein the body fluids are blood, plasma, serum, follicular fluid, synovial fluid, urea, milk, sweat, sperm, pulmonary fluid, saliva, secretions of the gastrointestinal tract and its appended glands, tear fluid, liquor and/or secretion products.

10. The method as claimed in one of the preceding claims, wherein a specific dilution step acts as pretreatment, which specific dilution step if appropriate, i.e. especially in the case of solid biological materials, is additionally combined with a disruption step.

11. The method as claimed in one of the preceding claims, which comprises a pretreatment with certain salt solutions or buffer solutions, in particular urea solutions, of differing concentration.

12. The method as claimed in claim 11, wherein the buffer solution contains an addition of water-soluble organic components, detergents, surfactants, in particular nonionic surfactants, or emulsifiers, DMSO or DTT or enzymes.

13. The method as claimed in one of the preceding claims, wherein, for the extraction, as organic solvent, use is made of polar protic solvents, in particular alcohols.

14. The method as claimed in claim 13, wherein the polar protic solvents are selected from the group consisting of methanol, ethanol, 1-propanol, 2-propanal (isopropanol), butanol, pentanol, hexanol and also mixtures thereof.

15. The method as claimed in one of the preceding claims, wherein, for the extraction, as solvent, use is made of at least one nonpolar solvent, in particular alkanes, preferably C5 to C12 alkanes.

16. The method as claimed in claim 15, wherein, as nonpolar solvent, use is made of hexane, heptane and/or octane, in particular isooctane, preferably mixtures thereof.

17. The method as claimed in one of the preceding claims, wherein the solvents are used in the form of solvent mixtures, in particular in the form of solvent mixtures which comprise polar and nonpolar solvents.

18. The method as claimed in one of the preceding claims, wherein the extracted components are analyzed spectrometrically, wherein spectrometric methods which come into consideration are those which examine the components by an interaction with electromagnetic radiation, in particular NMR, IR, UV-VIS, laser-Raman spectroscopy.

19. The method as claimed in one of the preceding claims, wherein the fat-soluble components are examined directly spectrometrically, in particular colorimetrically, preferably fluorimetrically.

20. The method as claimed in either of claim 18 or 19, wherein the fat-soluble components are examined directly spectrophotometrically.

21. The method as claimed in one of the preceding claims, wherein, before the analysis, the extracted components are enriched by means of a chromatographic method and if appropriate removed from accompanying substances which interfere with the analysis.

22. The method as claimed in one of the preceding claims, wherein the fat-soluble components are modified by a reaction and then examined directly spectrometrically, in particular calorimetrically, preferably fluorimetrically.

23. The use of the method as claimed in one of the preceding claims for analysis of components of biological materials, wherein the analysis is used for the detection of dyes in foods and feeds.

24. The use as claimed in claim 23, wherein the analysis is used for the pursuant of color falsifications in foods and feeds.

25. The use as claimed in claim 23 or 24 for analysis of carotenoids in fish.

26. The use as claimed in claim 23 or 24 for analysis of dyes in eggs or egg-containing products.

27. An analytical unit for carrying out the method as claimed in one of the preceding claims, consisting of a pretreatment kit and an extraction kit, wherein both kits contain one each of a solvent and/or solvent mixture of the above-defined type which is dependent on the biological material and the substance to be analyzed.

28. The analytical unit as claimed in claim 27 for detection of dyes in foods and feed.

29. The analytical unit as claimed in claim 28 for the spectrophotometric detection of carotenoids in foods, in particular egg and fish.

30. A spectrophotometer, in particular hand photometer, for measuring components of biological materials in a transparent vessel forming the extraction kit as claimed in claim 27, wherein the beam path of the photometer is adjusted in such a manner that the measurement records the upper half of the vessel.
